(19) United States
(12) Patent Application Publication
(10) Pub. No.: US 2012/0263848 A1
(43) Pub. Date: Oct. 18, 2012

(54) Organic Compounds

(76) Inventors: David C. Bom, Cincinnati, OH (US); Stefan Michael Furrer, Cincinnati, OH (US)

(21) Appl. No.: 13/503,174

(22) PCT Filed: Oct. 28, 2010
(86) PCT No.: PCT/EP2010/066368

§ 371 Date: May 15, 2012

Related U.S. Application Data
(60) Provisional application No. 61/256,489, filed on Oct. 30, 2009.
(60) Provisional application No. 61/388,270, filed on Sep. 30, 2010.

(51) Int. Cl. A23L 1/226; C07D 209/08

(52) U.S. Cl. 426/537; 548/510

(57) ABSTRACT

Organic compounds of formula (I),

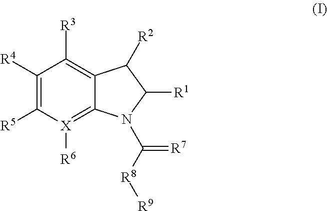

wherein:

X is C,

R¹ to R⁶ may be independently selected from the group consisting of, H, C₁-C₃ alkyl, Br, F, Cl,

R⁷ is O

R⁸ is NH

R⁹ is a group having 5 to 12 carbons selected from the group consisting of cycloalkyl or substituted alkyl, wherein the substituent is selected from the group consisting of aryl or cycloalkyl.

The compounds create/modify flavour, in particular sweet flavour and may be used in compositions and consumable products.

This invention relates to compounds that create enhance or modify flavour, in particularly sweet flavour, to methods of their manufacture and to compositions and consumable products containing said compounds.

In the flavour industry there is a constant demand for new compounds that create enhance or modify the flavour of compositions and consumable products.

Such compounds extend a flavourist's palette and result in greater product diversity for consumers.

In particular there is demand for compounds that are able to create or enhance or modify the sweet flavour of compositions and consumable products. Such compounds may replace, or reduce reliance on conventional sugars the use of which can be undesirable due to health and wellness concerns such as diabetes and obesity.

Furthermore, sweet-tasting materials may be useful for masking bitter tastes associated with certain compositions or consumable products, such as pharmaceutical compositions and products, leading to increased acceptance by users.

The present invention relates to compounds of formula (I), as hereinbelow defined, and to the use of such compounds to create, enhance or modify the flavour, in particular the sweet flavour of compositions and consumable products.

The present invention also relates to methods of creating enhancing, or modifying the flavour, in particular the sweet flavour of compositions and consumable products by adding to said compositions or consumable products at least one compound of formula (I).

Furthermore the present invention relates to compositions and consumable products comprising compounds of formula (I)

The term “consumable product” as used herein refers to composition that may be placed in the oral cavity and ingested or that may be placed in the oral cavity before being discarded for example mouthwash and chewing gum.

The present invention can be understood more readily by reference to the following detailed description of the various embodiments, and to the examples included herein.

In a first aspect of the present invention there is provided a compound of formula (I).

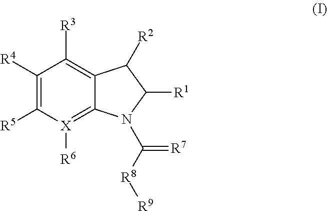

wherein:
X is C,
R¹ to R⁶ may be independently selected from the group consisting of, H, C₁-C₃ alkyl, Br, F, Cl,
R⁷ is O,
R⁸ is NH,
R⁹ is a group having 5 to 12 carbons selected from the group consisting of cycloalkyl, or substituted alkyl wherein the substituent is selected from the group consisting of aryl, cycloalkyl.

In a particular embodiment R¹ to R⁶ may be independently selected from the group consisting of, H, F, and Methyl.

In particular embodiment R⁹ is phenylethyl, phenylmethyl, cyclohexylmethyl, cyclohexyl, cycloheptyl, or cyclooctyl.

In a more particular embodiment compounds of formula (I) may be selected from the group consisting of: N-cycloheptylindoline-1-carboxamide; N-cycloheptyl-2-methylindoline-1-carboxamide; N-cyclooctylindoline-1-carboxamide; N-cyclooctyl-2-methylindoline-1-carboxamide; N-(cyclohexylmethyl)indoline-1-carboxamide; N-(cyclohexylmethyl)-2-methylindoline-1-carboxamide; N-cyclohexyl-3-methylindoline-1-carboxamide; N-cyclohexyl-6-fluoroindoline-1-carboxamide; N-cyclohexyl-5-fluoroindoline-1-carboxamide; N-cyclohexyl-4-methylindoline-1-carboxamide; N-benzylindoline-1-carboxamide.

Compounds of formula (I) may exist as a mixture of stereoisomers. Such stereoisomers of the compounds of formula (I) may be used as a racemic mixture, or the mixture may be resolved into its stereoisomers by techniques generally known in the art. The use of the term “a compound” of formula (I) may refer to both a racemic mixture and the individually isolated isomers.

Compounds of the formula (I) can be formed by known methods using commercially available starting materials, reagents and solvents.

The compounds of formula (I) may be prepared by a process comprising the steps of:
(a) Reduction of an appropriately substituted indole, heteroarylpyrolidine or heteroarylpyrrole derivative bearing groups R₁ to R₆ to provide an intermediate.
(b) Subsequent treatment of said intermediate with an appropriately substituted isocyanate or acid chloride bearing groups R₇ to R₉.

Non limiting examples of common reducing agents include pyridine borane, sodium cyanoborohydride, sodium borohydride, sodium triacetoxyborohydride and reagents commonly used for catalytic hydrogenation.

Scheme 1 shows particularly preferred methods of forming compounds of formula I.

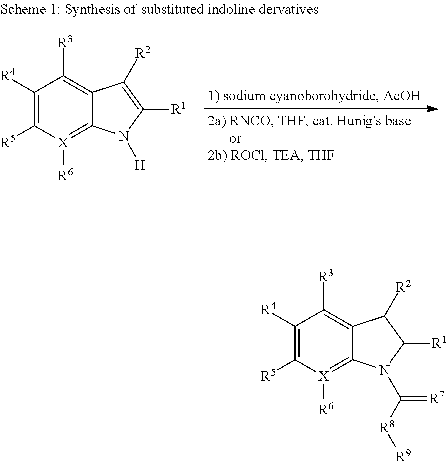

If desired, individual enantiomers of the compounds of formula (I) can be prepared.

The individual enantiomers can be prepared by a variety of known methods using commercially available starting materials, reagents and solvents. Non limiting examples of processes that can be used to prepare the individual enantiomers of compounds of formula (I) are given below.

The individual enantiomers can be prepared by a process comprising:
(a) An asymmetric reduction of an appropriately substituted indole or heteroarylpyrrole derivative bearing groups R₁ to R₆ to provide an intermediate,
(b) Subsequent treatment of said intermediate with an appropriately substituted isocyanate or acid chloride bearing groups R₇ to R₉

Alternatively the individual enantiomers of the compounds of formula (I) may be prepared by using the appropriately substituted pure enantiomeric form of the R or S substituted indoline or heteroarylpyrrolidine as starting materials in a process comprising:
(a) Reduction of an appropriately substituted enantiomeric form of indoline or heteroarylpyrrolidine derivative bearing groups R₁ to R₆ to provide an intermediate.
(b) Subsequent treatment of said intermediate with an appropriately substituted isocyanate or acid chloride bearing groups R₇ to R₉.

The pure enantiomeric form of the R or S substituted indolines and heteroarylpyrrolidines may be prepared using an approach described by Bertini-Gross et al. J. Org. Chem. Vol. 62, 1997, pp. 7679-7689, incorporated herein by reference.

Alternatively the individual enantiomers of the compounds of formula (I) may be prepared as shown in scheme 2. This process comprises
(c) Reduction of an appropriately substituted indole or heteroarylpyrrole derivative bearing groups R₁ to R₆ to provide an intermediate,
(d) Treatment of said intermediate with an appropriately substituted isocyanate or acid chloride bearing groups R₇ to R₉, and
b) Subsequent asymmetric reduction

Non limiting examples of common reducing agents for asymmetric reduction include hydrogen, a metal catalyst and a chiral ligand.

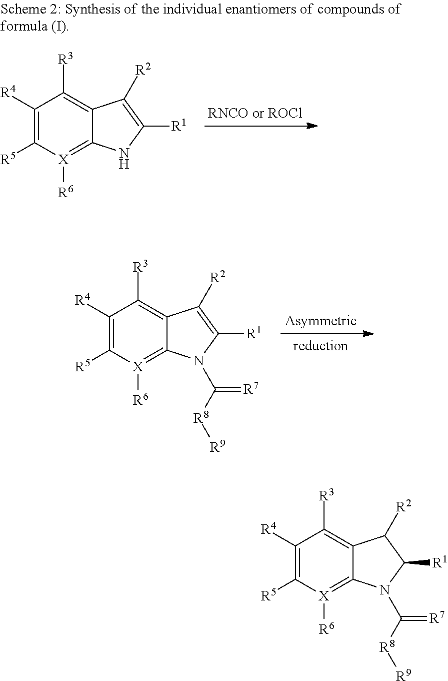

Alternatively the individual enantiomers of the compounds of formula (I) may be enzymatically or non-enzymatically resolved from a racemic solution. For an example of a non-enzymatic approach see Arp and Fu, J. Am. Chem. Soc., Vol. 128, 2006, pp. 14264-14265, incorporated herein by reference. For an example of an enzymatic approach see Gotor-Fernandez et al. Tetrahedron Asymmetry, Vol. 17, 2006, pp. 2558-2564, incorporated herein by reference.

Further examples of suitable reaction conditions are provided in the examples.

The applicant has found that compounds of formula (I) may be used to create or modify the flavour, in particular the sweet flavour, of compositions and consumable products.

Furthermore, the applicant has found that compounds of formula (I) may be used as sweet flavour enhancers to enhance the sweet flavour of compositions and consumable products comprising at least one other sweet flavoured compound.

The applicant has found that the R enantiomer of the compounds of formula (I) are particularly effective at creating modifying, or enhancing the sweet flavour of compositions and consumable products.

(R)-N-cyclooctyl-2-methylindoline-1-carboxamide and (R)-N-cycloheptyl-2-methylindoline-1-carboxamide have been found to be particularly effective at creating modifying, or enhancing the sweet flavour of compositions and consumable products.

Examples of sweet flavoured compounds are sweeteners and sweetener enhancers, non limiting examples of which include, but are not limited to, fructose, glucose, sucrose, lactose, maltose, saccharin, aspartame, sucralose, neotame, sorbitol, xylitol, maltodextrol, polyols, neohesperidin dihydrochalcone, rebaudioside, stevioside, neotame, mannitol, erithrytol, xylose, rhamnose, Luo Han Guo extract, mogriside (V), stevia extract, thaumatin, inositol, and trilobatin.

The term “sweet flavour enhancer”, as used herein, refers to a compound of the formula (I), that when used in a composition or consumable product comprising at least one other sweet flavoured compound results in a more than additive increase in the overall sweet flavour of the composition or consumable product.

In an aspect of the present invention there is provided a method of creating or modifying the flavour, in particular the sweet flavour, of a composition comprising the step of adding to said composition at least one compound of formula (I) as defined hereinabove.

In another aspect of the present invention there is provided a method of enhancing the sweet flavour of a composition comprising at least one other sweet flavoured compound, comprising the step of adding to said composition at least one compound of formula (I) as defined hereinabove.

In a particular embodiment there is provided a method of creating modifying, or enhancing the flavour, in particular the sweet flavour, of a composition comprising adding to said composition the R enantiomer of at least one compound of formula (I) as defined hereinabove.

In a particular embodiment there is provided a method of creating modifying, or enhancing the flavour, in particular the sweet flavour, of a composition comprising adding to said composition (R)-N-cyclooctyl-2-methylindoline-1-carboxamide and/or (R)-N-cycloheptyl-2-methylindoline-1-carboxamide.

The compounds of formula (I) may be added into a composition in neat form, or in a solvent, or they may first be modified, for example by entrapped with an entrapment material such as for example polymers, capsules, microcapsules, nanocapsules, liposomes, precursors, film formers, absorbents such as for example by using carbon or zeolites, cyclic oligosaccharides and mixtures thereof, or they may be chemically bound to substrates which are adapted to release the compounds of formula (I) upon application of an exogenous stimulus such as light, enzymes, or the like.

A compound of formula (I) may be used as the sole flavouring component in a composition. Alternatively a compound of formula (I) may be employed in conjunction with other compounds of formula (I) and/or other flavourant ingredients known in the art, in particularly sweeteners, and sweetener enhancers.

Compounds of formula (I) may be used in a compositions at a concentration of up to 100% by weight of the flavour components of the composition. However, more commonly compounds of formula (I) will be used with other flavour ingredients, in particular sweeteners and sweetener enhancers, at a concentration of 0.01% to 99.9% by weight of the flavour components of the composition.

Other flavourant ingredients that may be used with compounds of formula (I) include, but are not limited to, natural flavours, artificial flavours, spices, seasonings, and the like. Exemplary flavouring ingredients include synthetic flavour oils and flavouring aromatics and/or oils, oleoresins, essences, distillates, and extracts derived from plants, leaves, flowers, fruits, and so forth, and combinations comprising at least one of the foregoing.

Exemplary flavour oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavouring agents include artificial, natural and synthetic fruit flavours such as vanilla, and citrus oils including lemon, orange, lime, grapefruit, yazu, sudachi, and fruit essences including apple, pear, peach, grape, blueberry, strawberry, raspberry, cherry, plum, prune, raisin, cola, guarana, neroli, pineapple, apricot, banana, melon, apricot, ume, cherry, raspberry, blackberry, tropical fruit, mango, mangosteen, pomegranate, papaya and so forth. Additional exemplary flavours imparted by a flavouring agent include a milk flavour, a butter flavour, a cheese flavour, a cream flavour, and a yogurt flavour; a vanilla flavour; tea or coffee flavours, such as a green tea flavour, an oolong tea flavour, a tea flavour, a cocoa flavour, a chocolate flavour, and a coffee flavour; mint flavours, such as a peppermint flavour, a spearmint flavour, and a Japanese mint flavour; spicy flavours, such as an asafetida flavour, an ajowan flavour, an anise flavour, an angelica flavour, a fennel flavour, an allspice flavour, a cinnamon flavour, a chamomile flavour, a mustard flavour, a cardamom flavour, a caraway flavour, a cumin flavour, a clove flavour, a pepper flavour, a coriander flavour, a sassafras flavour, a savory flavour, a Zanthoxyli Fructus flavour, a perilla flavour, a juniper berry flavour, a ginger flavour, a star anise flavour, a horseradish flavour, a thyme flavour, a tarragon flavour, a dill flavour, a capsicum flavour, a nutmeg flavour, a basil flavour, a marjoram flavour, a rosemary flavour, a bayleaf flavour, and a wasabi (Japanese horseradish) flavour; a nut flavour such as an almond flavour, a hazelnut flavour, a macadamia nut flavour, a peanut flavour, a pecan flavour, a pistachio flavour, and a walnut flavour; alcoholic flavours, such as a wine flavour, a whisky flavour, a brandy flavour, a rum flavour, a gin flavour, and a liqueur flavour; floral flavours; and vegetable flavours, such as an onion flavour, a garlic flavour, a cabbage flavour, a carrot flavour, a celery flavour, mushroom flavour, and a tomato flavour.

In some embodiments, said other flavourant ingredients include aldehydes and esters such as cinnamyl acetate, cinnamaldehyde, citral diethylacetal, dihydrocarvyl acetate, eugenyl 49 formate, p-methylamisol, and so forth can be used. Further examples of aldehyde flavourings include acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavours), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), and the like.

Further examples of other flavourant ingredients can be found in “Chemicals Used in Food Processing”, publication 1274, pages 63-258, by the National Academy of Sciences.

Compounds of formula (I) can additionally be used in compositions, as described hereinabove, in conjunction with one or more ingredients or excipients conventionally used in compositions, for example carrier materials and other auxiliary agents commonly used in the art. Suitable excipients for compositions are well known in the art and include, for example, without limitation, solvents (including water, alcohol, ethanol, oils, fats, vegetable oil, and miglyol), binders, diluents, disintegrating agents, lubricants, flavouring agents, coloring agents, preservatives, antioxidants, emulsifiers, stabilisers, flavour-enhancers, anti-caking agents, and the like.

Examples of such carriers or diluents for compositions may be found in for example, “Perfume and Flavour Materials of Natural Origin”, S. Arctander, Ed., Elizabeth, N.J., 1960; in “Perfume and Flavour Chemicals”, S. Arctander, Ed., Vol. I & II, Allured Publishing Corporation, Carol Stream, USA, 1994; in “Flavourings”, E. Ziegler and H. Ziegler (ed.), Wiley-VCH Weinheim, 1998, and “CTFA Cosmetic Ingredient Handbook”, J. M. Nikitakis (ed.), 1st ed., The Cosmetic, Toiletry and Fragrance Association, Inc., Washington, 1988.

Other suitable and desirable ingredients of compositions are described in standard texts, such as “Handbook of Industrial Chemical Additives”, ed. M. and I. Ash, 2^{nd} Ed., (Synapse 2000).

In another aspect of the present invention there is provided a method of creating, or modifying the flavour, in particularly the sweet flavour, of a consumable product comprising the step of adding to said consumable product at least one compound of formula (I).

In another aspect of the present invention there is provided a method of enhancing the sweet flavour of a consumable product comprising at least one other sweet flavour compound, comprising the step of adding to said consumable product at least one compound of formula (I).

In a particular embodiment there is provided a method of creating modifying, or enhancing the flavour, in particular the sweet flavour, of a consumable product comprising adding to said composition the R enantiomer of at least one compound of formula (I) as defined hereinabove

In a particular embodiment there is provided a method of creating modifying, or enhancing the flavour, in particular the sweet flavour, of a consumable product comprising adding to said composition (R)-N-cyclooctyl-2-methylindoline-1-carboxamide and/or (R)-N-cycloheptyl-2-methylindoline-1-carboxamide.

Compounds of formula (I), or compositions comprising at least one compound of formula (I) can be added to consumable products by using conventional techniques to directly admix said compounds or compositions into the consumable product.

The quantities in which compounds of formula (I) may be added to consumable products may vary within wide limits and depend, inter alia, on the nature of the consumable product, on the effect desired, the purpose of adding compounds of formula (I) to a consumable product, for example masking a bitter taste, or enhancing a sweet taste, and on the nature and quantity of any other components of the consumable product.

Non limiting, concentrations of compounds of formula (I), in ppm by weight based on the weight of the consumable product, maybe: 500 ppm to 0.01 ppm, more particularly 250 ppm to 0.01 ppm, still more particularly 100 ppm to 1 ppm.

The compounds of formula (I) can be added to all manner of consumable products. Examples include, but are not limited to, foodstuffs of all kinds, confectionery products, baked products, sweet products, savory products, dairy products, beverages and oral care products.

Exemplary foodstuffs include, but are not limited to, chilled snacks, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, uht soup, frozen soup, pasta, canned pasta, dried pasta, chilled/fresh pasta, noodles, plain noodles, instant noodles, cups/bowl instant noodles, pouch instant noodles, chilled noodles, snack noodles, dried food, dessert mixes, sauces, dressings and condiments, herbs and spices, spreads, jams and preserves, honey, chocolate spreads, nut-based spreads, and yeast-based spreads.

Exemplary confectionery products include, but are not limited to, chewing gum (which includes sugarized gum, sugar-free gum, functional gum and bubble gum), centerfill confections, chocolate and other chocolate confectionery, medicated confectionery, lozenges, tablets, pastilles, mints, standard mints, power mints, chewy candies, hard candies, boiled candies, breath and other oral care films or strips, candy canes, lollipops, gummies, jellies, fudge, caramel, hard and soft panned goods, toffee, taffy, liquorice, gelatin candies, gum drops, jelly beans, nougats, fondants, combinations of one or more of the above, and edible compositions incorporating one or more of the above.

Exemplary baked products include, but are not limited to, alfajores, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes,

Exemplary sweet products include, but are not limited to, breakfast cereals, ready-to-eat (“rte”) cereals, family breakfast cereals, flakes, muesli, other rte cereals, children's breakfast cereals, hot cereals,

Exemplary savory products include, but are not limited to, salty snacks (potato chips, crisps, nuts, tortilla-tostada, pretzels, cheese snacks, corn snacks, potato-snacks, ready-to-eat popcorn, microwaveable popcorn, pork rinds, nuts, crackers, cracker snacks, breakfast cereals, meats, aspic, cured meats (ham, bacon), luncheon/breakfast meats (hotdogs, cold cuts, sausage), tomato products, margarine, peanut butter, soup (clear, canned, cream, instant, UHT), canned vegetables, pasta sauces.

Exemplary dairy products include, but are not limited to, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavoured, functional and other condensed milk, flavoured milk drinks, dairy only flavoured milk drinks, flavoured milk drinks with fruit juice, soy milk, sour milk drinks, fermented dairy drinks, coffee whiteners, powder milk, flavoured powder milk drinks, cream, yoghurt, plain/natural yoghurt, flavoured yoghurt, fruited yoghurt, probiotic yoghurt, drinking yoghurt, regular drinking yoghurt, probiotic drinking yoghurt, chilled and shelf-stable desserts, dairy-based desserts, soy-based desserts,

Exemplary beverages include, but are not limited to, flavoured water, soft drinks, fruit drinks, coffee-based drinks, tea-based drinks, juice-based drinks (includes fruit and vegetable), milk-based drinks, gel drinks, carbonated or non-carbonated drinks, powdered drinks, alcoholic or non-alcoholic drinks.

There now follows a series of non-limiting examples that serve to illustrate the invention.

### EXAMPLE 1

### N-cycloheptylindoline-1-carboxamide

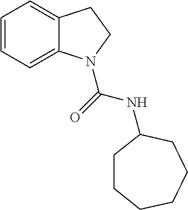

Indoline (0.362 ml, 3.23 mmol) was dissolved in THF (16 ml) and treated with Hunig's base (3 drops) followed by cycloheptyl isocyanate (0.428 ml, 3.23 mmol). After 2 h, the solvent was removed and the crude solid was dissolved in dichloromethane and purified by flash chromatography using a gradient of 100% hexane to 100% MTBE providing the above titled product (0.64 g, 77%) as a tan solid.

¹H NMR (CDCl₃; 300 MHz) δ 1.42-1.64 (m, 10 H), 1.96-2.01 (m, 2 H), 3.14 (t, J=9 Hz, 2 H), 3.86 (t, J=9 Hz, 2 H), 3.92-3.99 (m, 1 H), 4.50 (br d, J=7 Hz, 1 H), 6.88 (t, J=7 Hz, 1 H), 7.10-7.16 (m, 2 H), 7.86 (d, J=8 Hz, 1 H); ¹³C NMR (CDCl₃; 75 MHz) δ 24.3, 28.0, 28.2, 35.9, 47.2, 51.5, 114.7, 121.7, 124.7, 127.7, 130.4, 144.2, 154.7; MS calculated for C16H22N2O+H 259, observed 259.

### EXAMPLE 2

### N-cycloheptyl-2-methylindoline-1-carboxamide

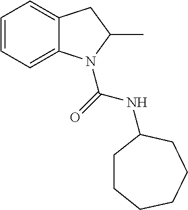

2-Methylindoline (0.421 ml, 3.23 mmol) was dissolved in THF (17 ml) and treated with Hunig's base (3 drops) followed by cycloheptyl isocyanate (0.428 ml, 3.23 mmol). After 2 h, the solvent was removed and the crude solid was dissolved in dichloromethane and purified by flash chromatography using a gradient of 100% hexane to 100% MTBE providing the above titled product (0.5 g, 57%) as a white solid.

¹H NMR (CDCl₃; 300 MHz) δ 1.30 (d, J=6 Hz, 3 H), 1.46-1.64 (m, 10 H), 1.96-2.04 (m, 2 H), 2.60 (dd, J₁=16 Hz, J₂=2 Hz, 1 H), 3.37 (dd, J₁=16 Hz, J₂=9 Hz, 1 H), 3.95-4.00 (m, 1 H), 4.34-4.41 (m, 1 H), 4.69 (br d, J=7 Hz, 1 H), 6.88-6.93 (m, 1 H), 7.13-7.18 (m, 2 H), 7.65 (d, 8 Hz, 1 H); ¹³C NMR (CDCl₃; 75 MHz) δ 21.2, 24.4, 28.2, 35.9, 35.91, 36.5, 51.5, 55.4, 114.8, 122.0, 125.4, 127.7, 130.1, 142.7, 154.0; MS calculated for C17H24N2O+H 273, observed 273.

EXAMPLE 3

### N-cyclooctylindoline-1-carboxamide

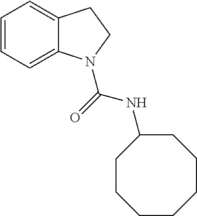

Indoline (0.367 ml, 3.26 mmol) was dissolved in THF (20 ml) and treated with Hunig's base (3 drops) followed by cyclooctylisocyanate (0.5 g, 3.26 mmol). After stirring overnight, the solvent was concentrated and the residue was dissolved in dichloromethane and purified by flash chromatography on SiO₂ using a gradient of 100% hexane to 50:50 hexane/MTBE providing the above titled product (0.5 g, 53%) as a white solid.

¹H NMR (CDCl₃; 300 MHz) δ 1.56-1.65 (m, 12 H), 1.88-1.95 (m, 2 H), 3.15 (t, J=9 Hz, 2 H), 3.88 (t, J=8 Hz, 2 H), 3.99-4.02 (m, 1 H), 4.49 (br d, J=7 Hz, 1 H), 6.85-6.91 (m, 1 H), 7.11-7.17 (m, 2 H), 7.85 (d, J=8 Hz, 1 H); ¹³C NMR (CDCl₃; 75 MHz) δ 24.0, 25.8, 27.4, 28.0, 33.2, 47.3, 50.3, 50.4, 114.7, 121.7, 124.7, 127.7, 130.4, 144.0, 154.5; MS calculated for C17H24N2O+H 273, observed 273.

### EXAMPLE 4

### N-cyclooctyl-2-methylindoline-1-carboxamide

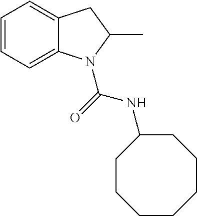

2-methylindoline (0.425 ml, 3.26 mmol) was dissolved in THF (20 ml) and treated with Hunig's base (3 drops) followed by cyclooctylisocyanate (0.5 g, 3.26 mmol). After stirring overnight, the solvent was concentrated and the residue was dissolved in dichloromethane and purified by flash chromatography on SiO₂ using a gradient of 100% hexane to 50:50 hexane/MTBE providing the above titled product (0.54 g, 51%) as a white solid.

¹H NMR (CDCl₃; 300 MHz) δ 1.30 (t, J=6 Hz, 3 H), 1.58-1.64 (m, 12 H), 1.88-1.98 (m, 2 H), 2.60 (dd, J₁=16 Hz, J₂=2 Hz, 1 H), 3.38 (dd, J₁=16 Hz, J₂=9 Hz, 1 H), 3.95-4.05 (m, 1 H), 4.33-4.43 (m, 1 H), 4.69 (br d, J=7 Hz, 1 H), 6.88-6.93 (m, 1 H), 7.13-7.19 (m, 2 H), 7.64 (d, J=8 Hz, 1 H); ¹³C NMR (CDCl₃; 75 MHz) δ 21.3, 24.0, 25.8, 27.4, 33.2, 36.5, 50.4, 55.4, 114.8, 122.0, 125.4, 127.7, 130.2, 142.7, 154.1; MS calculated for C18H26N2O+H 287, observed 287.

### EXAMPLE 5

### N-(cyclohexylmethyl)indoline-1-carboxamide

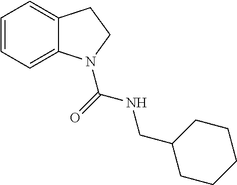

Indoline (0.404 ml, 3.59 mmol) was dissolved in THF (20 ml) and treated with Hunig's base (3 drops) followed by cyclohexanemethylisocyanate (0.51 ml, 3.59 mmol). After 2 h, the solvent was concentrated and the residue was dissolved in dichloromethane and purified by flash chromatography on SiO₂ using a gradient of 100% hexane to 50:50 hexane/MTBE providing the above titled product (0.79 g, 85%) as a white solid.

¹H NMR (CDCl₃; 300 MHz) δ 0.88-1.01 (m, 2 H), 1.08-1.32 (m, 3 H), 1.46-1.60 (m, 1 H), 1.68-1.80 (m, 5 H), 3.14-3.19 (m, 4 H), 3.90 (t, J=8 Hz, 2 H), 4.65 (br s, 1 H), 6.89 (ddd, J₁=7 Hz, J₂=1 Hz, 1 H), 7.12-7.18 (m, 2 H), 7.87 (d, J=8 Hz, 1 H); ¹³C NMR (CDCl₃; 75 MHz) δ 26.1, 26.7, 28.0, 31.1, 38.4, 46.9, 47.3, 114.8, 121.8, 124.7, 127.8, 130.4, 144.0, 155.4; MS calculated for C16H22N2O+H 259, observed 259.

### EXAMPLE 6

### N-(cyclohexylmethyl)-2-methylindoline-1-carboxamide

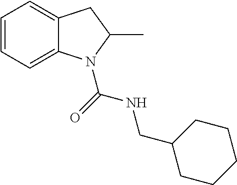

2-Methylindoline (0.468 ml, 3.59 mmol) was dissolved in THF (20 ml) and treated with Hunig's base (3 drops) followed by cyclohexanemethylisocyanate (0.51 ml, 3.59 mmol). After 2 h, the solvent was concentrated and the residue was dissolved in dichloromethane and purified by flash chromatography on SiO₂ using a gradient of 100% hexane to 50:50 hexane/MTBE providing the above titled product (0.68 g, 69%) as a white solid.

¹H NMR (CDCl₃; 300 MHz) δ 0.89-1.02 (m, 2 H), 1.13-1.28 (m, 3 H), 1.31 (d, J=6 Hz, 3 H), 1.49-1.62 (m, 1 H), 1.68-1.82 (m, 5 H), 2.61 (dd, J₁=16 Hz, J₂=2 Hz, 1 H), 3.15-3.21 (m, 2 H), 3.39 (dd, J₁=16 Hz, J₂=9 Hz, 1 H), 4.35-4.44 (m, 1 H), 4.83 (br s, 1 H), 6.91 (dd. J₁=7 Hz, J₂=1 Hz, 1 H), 7.14-7.19 (m, 2 H), 7.66 (d, J=8 Hz, 1 H); ¹³C NMR (CDCl₃; 75 MHz) δ 21.3, 26.1, 26.7, 31.2, 36.6, 38.5, 47.0, 55.4, 114.9, 122.1, 125.4, 127.8, 130.1, 142.7, 155.0; MS calculated for C17H24N2O+H 273, observed 273.

### EXAMPLE 7

### N-cyclohexyl-3-methylindoline-1-carboxamide

a) 3-methylindoline

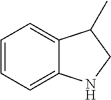

3-Methylindole (5 g, 38.2 mmol) was dissolved in glacial acetic acid (100 ml) and cooled to 0° C. Next, sodium cyanoborohydride (7.2 g, 114.6 mmol) was added portionwise. After 15 min, the reaction mixture was warmed to rt. After stirring for 3 h, the reaction was carefully poured into ice water (400 ml) and treated with 50% sodium hydroxide until the solution was strongly basic. Next, this mixture was extracted with MTBE (300 ml) and the organic extract was washed with water (2×150 ml), brine (150 ml) and dried (K₂CO₃). This product was carried forward without further purification. Structure and purity was confirmed by GC/MS. The retention time was 4.01 min. The MS was calculated for C9H11N 133, observed 133.

b) N-cyclohexyl-3-methylindoline-1-carboxamide

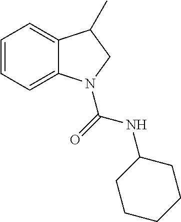

3-Methylindoline (1 g, 7.52 mmol) was dissolved in THF (38 ml) and treated with Hunig's base (3 drops) followed by cyclohexylisocyanate (0.96 ml, 7.52 mmol). After stirring overnight, the solvent was concentrated and the residue was dissolved in dichloromethane and purified by flash chromatography on SiO₂ using a gradient of 100% hexane to 50:50 hexane/MTBE providing the above titled product (1.45 g, 75%) as a white solid.

¹H NMR (CDCl₃; 300 MHz) δ 1.10-1.25 (m, 3 H), 1.34 (d, J=7 Hz, 3 H), 1.36-1.47 (m, 2 H), 1.59-1.78 (m, 3 H), 1.98-2.04 (m, 2 H), 3.38-3.50 (m, 2 H), 3.71-3.81 (m, 1 H), 4.04 (t, J=9 Hz, 1 H), 4.44 (br d, J=7 Hz, 1 H), 6.91 (ddd, J₁=7 Hz, J₂=1 Hz, 1 H), 7.10-7.18 (m, 2 H), 7.85 (d, J=8 Hz, 1 H); ¹³C NMR (CDCl₃; 75 MHz) δ 20.4, 25.2, 25.8, 34.0, 34.8, 49.2, 55.4, 114.6, 121.8, 123.5, 127.9, 135.6, 143.5, 154.5; MS calculated for C16H22N2O+H 259, observed 259.

### EXAMPLE 8

### N-cyclohexyl-6-fluoroindoline-1-carboxamide

a) 6-fluoroindoline

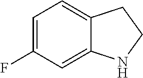

6-fluoroindole (5 g, 37 mmol) was dissolved in glacial acetic acid (100 ml) and cooled to 0° C. Next, sodium cyanoborohydride (7 g, 111 mmol) was added portionwise. After 15 min, the reaction mixture was warmed to rt. After stirring for 3 h, the reaction was carefully poured into ice water (400 ml) and treated with 50% sodium hydroxide until the solution was strongly basic. Next, this mixture was extracted with MTBE (300 ml) and the organic extract was washed with water (2×150 ml), brine (150 ml) and dried (K₂CO₃). This product was carried forward without further purification. Structure and purity was confirmed by GC/MS. The retention time was 3.96 min. The MS was calculated for C9H11N 137, observed 137.

b) N-cyclohexyl-6-fluoroindoline-1-carboxamide

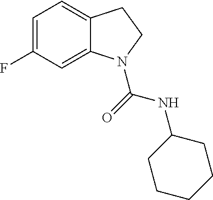

6-fluoroindoline (1 g, 7.30 mmol) was dissolved in THF (38 ml) and treated with Hunig's base (3 drops) followed by cyclohexylisocyanate (0.93 ml, 7.30 mmol). After stirring overnight, the solvent was concentrated and the residue was dissolved in dichloromethane and purified by flash chromatography on SiO₂ using a gradient of 100% hexane to 50:50 hexane/MTBE providing the above titled product (1.12 g, 58%) as a white solid.

¹H NMR (CDCl₃; 300 MHz) δ 1.09-1.25 (m, 3 H), 1.30-1.47 (m, 2 H), 1.58-1.78 (m, 3 H), 1.94-2.05 (m, 2 H), 3.11 (t, J=9 Hz, 2 H), 3.65-3.80 (m, 1 H), 3.89 (t, J=9 Hz, 2 H), 4.42 (br d, J=7 Hz, 1 H), 6.52-6.58 (m, 1 H), 6.94-7.02 (m, 1 H), 7.68 (dd, J₁=11 Hz, J₂=2 Hz, 1 H); ¹³C NMR (CDCl₃; 75 MHz) δ 25.1, 25.7, 27.3, 33.9, 47.9, 49.3, 103.2 (d, J=117 Hz), 107.8 (d, J=91 Hz), 124.7 (d, J=40 Hz), 125.3 (d, J=10 Hz), 145.4 (d, J=51 Hz), 154.3, 161.2, 164.4; MS calculated for C15H19FN2O+H263, observed 263.

### EXAMPLE 9

### N-cyclohexyl-5-fluoroindoline-1-carboxamide

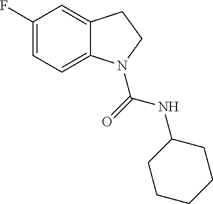

5-fluoroindoline (0.266 g, 1.94 mmol) was dissolved in THF (10 ml) and treated with Hunig's base (3 drops) followed by cyclohexylisocyanate (0.26 ml, 2.04 mmol). After stirring overnight, the solvent was concentrated and the residue was dissolved in dichloromethane and purified by flash chromatography on SiO₂ using a gradient of 100% hexane to 50:50 hexane/MTBE providing the above titled product (0.38 g, 75%) as a pinkish white solid.

¹NMR (CDCl₃; 300 MHz) δ 1.07-1.25 (m, 3 H), 1.33-1.48 (m, 2 H), 1.59-1.76 (m, 3 H), 1.95-2.04 (m, 2 H), 3.15 (t, J=9 Hz, 2 H), 3.68-3.81 (m, 1 H), 3.90 (t, J=9 Hz, 2 H), 4.33 (br d, J=7 Hz, 1 H), 6.80-6.87 (m, 2 H), 7.85 (dd, J₁=10 Hz, J₂=5 Hz, 1 H); ¹³C NMR (CDCl₃; 75 MHz) δ 111.9 (d, J=96 Hz), 113.8 (d, J=90 Hz), 115.5 (d, J=32 Hz), 131.9 (d, J=32 Hz), 140.2, 154.5, 156.9, 160.0; MS calculated for C15H19FN2O+H 263 observed 263.

### EXAMPLE 10

### N-cyclohexyl-4-methylindoline-1-carboxamide

a.) 4-methylindoline

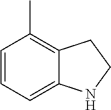

4-Methylindole (5 g, 38 mmol) was dissolved in glacial acetic acid (100 ml) and cooled to 0° C. Next, sodium cyanoborohydride (7.2 g, 114.5 mmol) was added portionwise. After 15 min, the reaction mixture was warmed to rt. After stirring for 3 h, the reaction was carefully poured into ice water (400 ml) and treated with 50% sodium hydroxide until the solution was strongly basic. Next, this mixture was extracted with MTBE (300 ml) and the organic extract was washed with water (2×150 ml), brine (150 ml) and dried (K₂CO₃). This product was carried forward without further purification. Structure and purity was confirmed by GC/MS. The retention time was 4.62 min. The MS was calculated for C9H11N 133, observed 133.

b) N-cyclohexyl-4-methylindoline-1-carboxamide

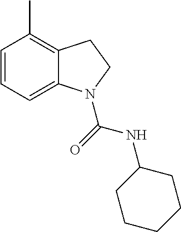

3-Methylindoline (1 g, 7.52 mmol) was dissolved in THF (10 ml) and treated with Hunig's base (3 drops) followed by cyclohexylisocyanate (0.96 ml, 7.52 mmol). After stirring overnight, the solvent was concentrated and the residue was dissolved in dichloromethane and purified by flash chromatography on SiO₂ using a gradient of 100% hexane to 50:50 hexane/MTBE providing the above titled product (1.49 g, 77%) as a white solid.

¹H NMR (CDCl₃; 300 MHz) δ 1.03-1.27 (m, 3 H), 1.32-1.49 (m, 2H), 1.55-1.78 (m, 3 H), 1.95-2.06 (m, 2 H), 2.19 (s, 3 H), 3.02 (t, J=9 Hz, 2 H), 3.68-3.82 (m, 1 H), 3.88 (t, J=8 Hz, 2 H), 4.48 (br d, J=7 Hz, 1 H), 6.71 (d, J=8 Hz, 1 H), 7.06 (t, J=8 Hz, 1 H), 7.67 (d, J=8 Hz, 1 H); ¹³C NMR (CDCl₃; 75 MHz) δ 18.7, 25.2, 25.8, 26.7, 34.0, 47.1, 49.2, 112.1, 122.8, 127.8, 129.2, 134.0, 143.6, 154.5; MS calculated for C16H22N2O+H 259, observed 259.

### EXAMPLE 11

### N-benzylindoline-1-carboxamide

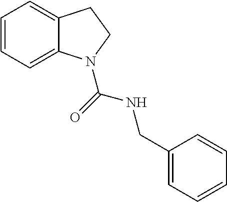

Indoline (0.941 ml, 8.39 mmol) was dissolved in THF (42 ml) and treated with Hunig's base (3 drops) followed by benzyl isocyanate (1.08 ml, 8.81 mmol). After stirring overnight, the solvent was concentrated and the residue was dissolved in dichloromethane and purified by flash chromatography on SiO₂ using a gradient of 100% hexane to 50:50 hexane/MTBE providing the above titled product (1.78 g, 84%) as a white solid.

¹ NMR (CDCl₃; 300 MHz) δ 3.15 (t, 9 Hz, 2 H), 3.89 (t, J=9 Hz, 2 H), 4.51 (d, J=6 Hz, 2 H), 4.93 (br s, 1 H), 6.90 (ddd, J₁=7 Hz, J₂=1 Hz, 1 H), 7.12-7.18 (m, 2 H), 7.25-7.30 (m, 1 H), 7.33-7.35 (m, 4 H), 7.91 (d, J=8 Hz, 1 H); ¹³C NMR (CDCl₃; 75 MHz) δ 28.0, 44.7, 47.3, 114.9, 122.0, 124.7, 127.6, 127.8. 127.9, 128.9, 130.4, 139.3, 143.8, 155.1; MS calculated for C16H16N2O+H 253, observed 253.

### EXAMPLE 12

### N-phenethylindoline-1-carboxamide

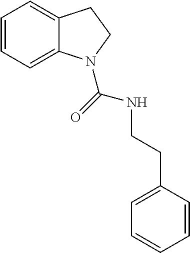

Indoline (0.943 ml, 8.39 mmol) was dissolved in THF (40 ml) and treated with Hunig's base (3 drops) followed by (2-isocyanatoethyl)benzene (1.16 ml, 8.39 mmol). After stirring overnight, the solvent was concentrated and the residue was dissolved in dichloromethane and purified by flash chromatography on SiO₂ using a gradient of 100% hexane to 50:50 hexane/MTBE providing the above titled product (1.69 g, 73%) as a white solid.

¹H NMR (CDCl₃; 300 MHz) δ 2.88 (t, J=7 Hz, 2 H), 3.10 (t, J=9 Hz, 2 H), 3.58 (dd, J₁=13 Hz, J₂=7 Hz, 2 H), 3.75 (t, J=9 Hz, 2 H), 4.61 (br t, J=5 Hz, 1 H), 6.88 (ddd, J₁=7 Hz, J₂=1 Hz, 1 H), 7.10-7.13 (m, 2 H), 7.20-7.25 (m, 3 H), 7.29-7.34 (m, 2 H), 7.83 (d, J=8 Hz, 1 H): ¹³C NMR (CDCl₃; 75 MHz) δ 27.9, 36.4, 41.8, 47.0, 114.7, 121.8, 124.7, 126.6, 127.7, 128.8, 129.0, 130.3, 139.4, 144.0, 155.4; MS calculated for C17H18N2O+H 267, observed 267.

### EXAMPLE 13

### 2-methyl-N-phenethylindoline-1-carboxamide

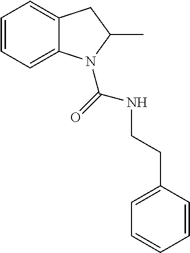

2-Methylindoline (0.978 ml, 7.51 mmol) was dissolved in THF (40 ml) and treated with Hunig's base (3 drops) followed by (2-isocyanatoethyl)benzene (1.10 ml, 7.51 mmol). After stirring overnight, the solvent was concentrated and the residue was dissolved in dichloromethane and purified by flash chromatography on SiO₂ using a gradient of 100% hexane to 50:50 hexane/MTBE providing the above titled product (1.7 g, 74%) as a white solid

¹H NMR (CDCl₃; 300 MHz) δ 1.19 (d, J=6 Hz, 3 H), 2.56 (dd, J₁=16 Hz, J₂=2 Hz, 1 H), 2.90 (t, J=7 Hz, 2 H), 3.33 (dd, J₁=16 Hz, J₂=9 Hz, 1 H), 3.57-3.64 (m, 2 H), 4.22-4.31 (m, 1 H), 4.75 (br t, J=5 Hz, 1 H), 6.90 (ddd, J₁=7 Hz, J₂=1 Hz, 1 H), 7.09-7.14 (m, 2 H), 7.20-7.26 (m, 3 H), 7.29-7.35 (m, 2 H), 7.54-7.57 (m, 1 H); ¹³C NMR (CDCl₃; 75 MHz) δ 21.1, 36.3, 36.5, 41.8, 55.3, 114.9, 122.1, 125.3, 126.7, 127.7, 128.8, 129.0, 130.0, 139.4, 142.5, 154.8; MS calculated for C18H20O+H 281, observed 281.

### EXAMPLE 14

### Taste Evaluation of Compounds Synthesized in Examples 1-13

1% solutions of compounds of formula (I) were prepared in ethanol.

These 1% solutions were then further diluted, using water, to create sample solutions of differing ppm concentrations.

A panel of trained experts was then asked to evaluate the taste of each sample solution and to comment on any inherent sweetness, other perceived taste, or flavor attributes.

After tasting each sample the experts rinsed their mouths with water and a break was observed until the stimulus disappeared.

The sample solutions were found to be sweet.

### EXAMPLE 15

### Isointensity Measurement of N-cycloheptyl-2-methylindoline-1-carboxamide

A 1% solution of N-cycloheptyl-2-methylindoline-1-carboxamide was prepared in ethanol.

This 1% solution was then further diluted, using water, to create a 5 ppm sample solution of N-cycloheptyl-2-methylindoline-1-carboxamide.

To determine the isointensity of this solution a panel of trained experts was then asked to evaluate the taste (in particularly the sweet taste) of this sample solution in comparison to 0.5%, 1% and 1.5% sample sucrose solutions.

Panel testing was carried out in the following way:

Twenty milliliters of each of the samples was presented blind and in counter balanced order following a William's Latin square design, to 20 sweet sensitive panelists.

In two replications (over 1 session), panelists were asked to taste and rank the solutions from least sweet to most sweet.

The R-index statistics were then calculated between the 5 ppm N-cycloheptyl-2-methylindoline-1-carboxamide sample solutions, and each of the sample sucrose solutions (0.5%, 1%, and 1.5%) to determine if they were significantly different from each other.

Samples were deemed significantly different if p<0.05 between the calculated R-index values and the critical value range of 37.76% -62.24%.

An R-index significantly greater than the critical value range means that the 5 ppm N-cycloheptyl-2-methylindoline-1-carboxamide sample solution is significantly less sweet than the sucrose sample. An R-index significantly below the lower critical value range indicates that the 5 ppm N-cycloheptyl-2-methylindoline-1-carboxamide sample solution is significantly sweeter than the sucrose sample.

Results are detailed in table I.

Based on these results it was determined that the 5 ppm N-cycloheptyl-2-methylindoline-1-carboxamide sample solution was iso-sweet to the 0.5% sucrose sample solution, and significantly less sweet than the 1.0% and 1.5% sucrose sample solutions (the calculated R-index values exceeding the critical values at p<0.05).

TABLE I
Isointensity data for 5 ppm N-cycloheptyl-2-methylindoline-1-
carboxamide sample solution.

| | | Critical | | |
|---|---|---|---|---|
| | | Value | | |
| | | Range (Non- | Significantly | Experimental |
| | R- | significant | different | Compound |
| Solutions compared | index | range) | (p < 0.05) | Sweetness |
| 5 ppm ppm N- | 39.38 | 37.76% to | No | Iso-sweet |
| cycloheptyl-2- | | 62.24% | | |
| methylindoline-1- | | | | |
| carboxamide vs. 0.5% | | | | |
| sucrose solution | | | | |
| 5 ppm N-cycloheptyl- | 78.63 | 37.76% to | Yes | Less sweet |
| 2-methylindoline-1- | | 62.24% | | |
| carboxamide vs. 1.0% | | | | |
| sucrose solution | | | | |
| 5 ppm N-cycloheptyl- | 92.00 | 37.76% to | Yes | Less sweet |
| 2-methylindoline-1- | | 62.24% | | |
| carboxamide vs. 1.5% | | | | |
| sucrose solution | | | | |

### EXAMPLE 16

### Sweet Enhancement in a Cola Beverage Application

A 1% ethanol solution of N-cycloheptyl-2-methylindoline-1-carboxamide was added into a 7% sucrose containing cola solution. This resulted in a 7% sucrose sample cola solution containing 5 ppm of N-cycloheptyl-2-methylindoline-1-carboxamide.

To determine its isointensity a panel of trained experts was then asked to evaluate the taste (in particularly the sweet taste) of this sample solution in comparison to sample cola solutions containing 7%, 8%, 9% and 10% sucrose but no N-cycloheptyl-2-methylindoline-1-carboxamide.

Panel testing was carried out in the following way:

Twenty milliliters of each of the sample solutions was presented blind and in random order to 20 sweet sensitive panelists.

In two replications (over 1 session), panelists were asked to rank the solutions from least sweet to most sweet.

The data was then subjected to an R-index analysis as described in example 15.

Results are detailed in table II.

TABLE II
Sweet enhancement results for 5 ppm N-cycloheptyl-2-methylindoline-
1-carboxamide in a cola application

| | | Critical | | |
|---|---|---|---|---|
| | | Value | | |
| | | Range (Non- | Significantly | Experimental |
| | R- | significant | different | Compound |
| Samples compared | index | range) | (p < 0.05) | Sweetness |
| 7% sugar + 5 ppm N- | 37.72 | 37.76% to | Yes | More sweet |
| cycloheptyl-2- | | 62.24% | | |
| methylindoline-1- | | | | |
| carboxamide vs. | | | | |
| 7% sugar | | | | |
| 7% sugar + 5 ppm N- | 40.03 | 37.76% to | No | Iso sweet |
| cycloheptyl-2- | | 62.24% | | |
| methylindoline-1- | | | | |
| carboxamide vs. | | | | |
| 8% sugar | | | | |
| 7% sugar + 5 ppm N- | 68.97 | 37.76% to | Yes | Less sweet |
| cycloheptyl-2- | | 62.24% | | |
| methylindoline-1- | | | | |
| carboxamide vs. | | | | |
| 9% sugar | | | | |
| 7% sugar + 5 ppm N- | 90.78 | 37.76% to | Yes | Less sweet |
| cycloheptyl-2- | | 62.24% | | |
| methylindoline-1- | | | | |
| carboxamide | | | | |
| vs. 10% sugar | | | | |

From the results it was determined that the 7% sucrose sample cola solution containing 5 ppm of N-cycloheptyl-2-methylindoline-1-carboxamide, was perceived as significantly more sweet than the 7% sugar cola solution, significantly less sweet than the 9% and 10% sugar cola solutions (the calculated R-index values exceeding the critical value range and p<0.05) and iso sweet to the 8% sugar cola solution.

Because in example 15 a 5 ppm N-cycloheptyl-2-methylindoline-1-carboxamide solution was perceived as iso sweet to a 0.5% sucrose solution, it would be expected that a 7% sucrose sample cola solution containing 5 ppm of N-cycloheptyl-2-methylindoline-1-carboxamide would be iso sweet to a 7.5% sucrose solution.

However, the results detailed in table II indicate that a 7% sucrose sample cola solution containing 5 ppm of N-cycloheptyl-2-methylindoline-1-carboxamide is iso sweet with an 8% sucrose solution. Consequently it may be concluded that cycloheptyl-2-methylindoline-1-carboxamide is behaving as a sweetness enhancer.

### EXAMPLE 17

### (S)-N-cycloheptyl-2-methylindoline-1-carboxamide

(S)-2-methylindoline (0.25 g, 1.88 mmol) was added to a 4 dram vial followed by THF (5 ml), Hunig's base (3 drops) and cycloheptylisocyanate (0.25 ml, 1.88 mmol). The contents were stirred overnight at 22° C. The reaction mixture was injected directly onto an 80 g SiO₂ and eluted with a gradient of 100% hexane to 70:30 hexane:EtOAc. Concentration of the product containing fractions provided the above titled product (0.33 g, 64%) as a light pink solid. Note: (S)-2-methylindoline was prepared in 100% ee following the four step procedure described by Bertini-Gross et al. J. Org. Chem. Vol. 62, 1997, pp. 7679-7689.

¹NMR (CDCl₃; 300 MHz) [symbol] 1.30 (d, J=6 Hz, 3 H), 1.42-1.64 (m, 10 H), 1.96-2.04 (m, 2 H), 2.60 (dd, J₁=16 Hz, J₂=2 Hz, 1 H), 3.38 (dd, J₁=15.8 Hz, J₂=9 Hz, 1 H), 3.93-4.03 (m, 1 H), 4.33-4.43 (m, 1 H), 4.68 (br d, J=7 Hz, 1 H), 6.88-6.93 (m, 1 H), 7.13-7.19 (m, 2 H), 7.63-7.66 (d, J=8 Hz, 1 H); MS calculated for C17H24N2O+H 273, observed 273.

### EXAMPLE 18

### (R)-N-cycloheptyl-2-methylindoline-1-carboxamide

(R)-2-methylindoline (0.25 g, 1.88 mmol) was added to a 4 dram vial followed by THF (5 ml), Hunig's base (3 drops) and cycloheptylisocyanate (0.25 ml, 1.88 mmol). The contents were stirred overnight at 22° C. The reaction mixture was injected directly onto an 80 g SiO₂ and eluted with a gradient of 100% hexane to 70:30 hexane:EtOAc. Concentration of the product containing fractions provided the above titled product (0.23 g, 45%) as a white solid. Note: (R)-2-methylindoline was prepared in 100% ee following the four step procedure described by Bertini-Gross et al. J. Org. Chem. Vol. 62, 1997, pp. 7679-7689.

¹H NMR (CDCl₃; 300 MHz) [symbol] 1.30 (d, J=6 Hz, 3 H), 1.42-1.64 (m, 10 H), 1.96-2.04 (m, 2 H), 2.60 (dd, J₁=16 Hz, J₂=2 Hz, 1 H), 3.38 (dd, J₁=15.8 Hz, J₂=9 Hz, 1 H), 3.93-4.03 (m, 1 H), 4.33-4.43 (m, 1 H), 4.68 (br d, J=7 Hz, 1 H), 6.88-6.93 (m, 1 H), 7.13-7.19 (m, 2 H), 7.63-7.66 (d, J=8 Hz, 1 H); MS calculated for C17H24N2O+H 273, observed 273.

### EXAMPLE 19

### (S)-N-cyclooctyl-2-methylindoline-1-carboxamide

(S)-2-methylindoline (0.25 g, 1.88 mmol) was added to a 4 dram vial followed by THF (5 ml), Hunig's base (3 drops) and cyclooctylisocyanate (0.29 ml, 1.88 mmol). The contents were stirred overnight at 22° C. The reaction mixture was injected directly onto an 80 g SiO₂ and eluted with a gradient of 100% hexane to 70:30 hexane:EtOAc. Concentration of the product containing fractions provided the above titled product (0.33 g, 61%) as a light pink solid. Note: (S)-2-methylindoline was prepared in 100% ee following the four step procedure described by Bertini-Gross et al. J. Org. Chem. Vol. 62, 1997, pp. 7679-7689.

¹H NMR (CDCl₃; 300 MHz) [symbol] 1.30 (d, J=6 Hz, 3 H), 1.58-1.64 (m, 12 H), 1.88-1.98 (m, 2 H), 2.60 (dd, J₁=16 Hz, J₂=2 Hz, 1 H), 3.38 (dd, J₁=16 Hz, J₂=9 Hz, 1 H), 4.01-4.05 (m, 1 H), 4.33-4.44 (m, 1 H), 4.69 (d, J=7 Hz, 1 H), 6.88-6.93 (m, 1 H), 7.14-7.19 (m, 2 H), 7.64 (d, J=8 Hz, 1 H); MS calculated for C18H26N2O+H 287, observed 287.

### EXAMPLE 20

### (R)-N-cyclooctyl-2-methylindoline-1-carboxamide

(R)-2-methylindoline (0.25 g, 1.88 mmol) was added to a 4 dram vial followed by THF (5 ml), Hunig's base (3 drops) and cyclooctylisocyanate (0.29 ml, 1.88 mmol). The contents were stirred overnight at 22° C. The reaction mixture was injected directly onto an 80 g SiO₂ and eluted with a gradient of 100% hexane to 70:30 hexane:EtOAc. Concentration of the product containing fractions provided the above titled product (0.24 g, 44%) as a light pink solid. Note: (R)-2-methylindoline was prepared in 100% ee following the four step procedure described by Bertini-Gross et al. J. Org. Chem. Vol. 62, 1997, pp. 7679-7689.

¹H NMR (CDCl₃; 300 MHz) [symbol] 1.30 (d, J=6 Hz, 3 H), 1.58-1.64 (m, 12 H), 1.88-1.98 (m, 2 H), 2.60 (dd, J₁=16 Hz, J₂=2 Hz, 1 H), 3.38 (dd, J₁=16 Hz, J₂=9 Hz, 1 H), 4.01-4.05 (m, 1 H), 4.33-4.44 (m, 1 H), 4.69 (d, J=7 Hz, 1 H), 6.88-6.93 (m, 1 H), 7.14-7.19 (m, 2 H), 7.64 (d, J=8 Hz, 1 H); MS calculated for C18H26N2O+H 287, observed 287.

### EXAMPLE 21

### Taste Evaluation of Compounds Synthesized in Examples 17-20

1% solutions of compounds of formula (I) were prepared in ethanol.

These 1% solutions were then further diluted, using water, to create sample solutions of differing ppm concentrations.

A panel of trained experts was then asked to evaluate the taste of each sample solution and to comment on any inherent sweetness, other perceived taste, or flavor attributes.

After tasting each sample the experts rinsed their mouths with water and a break was observed until the stimulus disappeared.

The sample solutions for examples 18 and 20 were found to be sweet.

### EXAMPLE 22

### Isointensity Measurement for (R)-N-cyclooctyl-2-methylindoline-1-carboxamide

A 1% solution of (R)-N-cyclooctyl-2-methylindoline-1-carboxamide was prepared in ethanol.

This 1% solution was then further diluted, using water, to create a 2 ppm sample solution of (R)-N-cyclooctyl-2-methylindoline-1-carboxamide. To determine the isointensity of this solution a panel of trained experts was then asked to evaluate the taste (in particularly the sweet taste) of this sample solution in comparison to 0.5%, 1% and 1.5% sample sucrose solutions.

Panel testing was carried out in the following way:

Twenty milliliters of each of the samples was presented blind and in counter balanced order following a William's Latin square design, to 20 sweet sensitive panelists.

In two replications (over 1 session), panelists were asked to taste and rank the solutions from least sweet to most sweet.

The R-index statistics were then calculated between the 2 ppm (R)-N-cyclooctyl-2-methylindoline-1-carboxamide sample solutions, and each of the sample sucrose solutions (0.5%, 1%, and 1.5%) to determine if they were significantly different from each other.

Samples were deemed significantly different if p<0.05 between the calculated R-index values and the critical value range of 37.76% -62.24%.

An R-index significantly greater than the critical value range means that the 2 ppm (R)-N-cyclooctyl-2-methylindoline-1-carboxamide sample solution is significantly less sweet than the sucrose sample. An R-index significantly below the lower critical value range indicates that the 2 ppm (R)-N-cyclooctyl-2-methylindoline-1-carboxamide sample solution is significantly sweeter than the sucrose sample.

Results are detailed in table I.

Based on these results it was determined that the 2 ppm (R)-N-cyclooctyl-2-methylindoline-1-carboxamide sample solution was iso-sweet to the 0.5% sucrose sample solution, and significantly less sweet than the 1.0% and 1.5% sucrose sample solutions (the calculated R-index values exceeding the critical values at p<0.05).

TABLE I
Isointensity data for 2 ppm (R)-N-cyclooctyl-2-methylindoline-1-
carboxamide sample solution.

| | | Critical | | |
|---|---|---|---|---|
| | | Value | Signifi- | |
| | | Range (Non- | cantly | Experimental |
| | R- | significant | different | Compound |
| Solutions compared | index | range) | (p < 0.05) | Sweetness |
| 2 ppm (R)-N-cyclooctyl- | 27.00 | 37.76% to | Yes | More sweet |
| 2-methylindoline- | | 62.24% | | |
| 1-carboxamide vs. | | | | |
| 0.5% sucrose solution | | | | |
| 2 ppm (R)-N-cyclooctyl- | 72.31 | 37.76% to | Yes | Less sweet |
| 2-methylindoline- | | 62.24% | | |
| 1-carboxamide vs. | | | | |
| 1.0% sucrose solution | | | | |
| 2 ppm (R)-N-cyclooctyl- | 99.13 | 37.76% to | Yes | Less sweet |
| 2-methylindoline- | | 62.24% | | |
| 1-carboxamide vs. | | | | |
| 1.5% sucrose solution | | | | |

### EXAMPLE 23

### Sweet Enhancement of (R)-N-cyclooctyl-2-methylindoline-1-carboxamide in 7% sucrose

A 1% ethanol solution of (R)-N-cyclooctyl-2-methylindoline-1-carboxamide was added into an aqueous 7% sucrose solution. This resulted in a 7% aqueous sucrose containing 2 ppm of (R)-N-cyclooctyl-2-methylindoline-1-carboxamide.

To determine its isointensity a panel of trained experts was then asked to evaluate the taste (in particularly the sweet taste) of this sample solution in comparison to aqueous sample solutions containing 7%, 8%, 9% and 10% sucrose, but without added (R)-N-cyclooctyl-2-methylindoline-1-carboxamide.

Panel testing was carried out in the following way:

Twenty milliliters of each of the sample solutions was presented blind and in random order to 20 sweet sensitive panelists.

In two replications (over 1 session), panelists were asked to rank the solutions from least sweet to most sweet.

The data was then subjected to an R-index analysis as described in example 5.

Results are detailed in table II.

TABLE II
Sweet enhancement results for 2 ppm (R)-N-cyclooctyl-2-methylindoline-
1-carboxamide in a cola application

| | | Critical Value | | |
|---|---|---|---|---|
| | | Range (Non- | Significantly | Experimental |
| | R- | significant | different | Compound |
| Samples compared | index | range) | (p < 0.05) | Sweetness |
| 7% sugar + 2 ppm | 35.53 | 37.76% to | Yes | More sweet |
| (R)-N-cyclooctyl-2- | | 62.24% | | |
| methylindoline-1- | | | | |
| carboxamide vs. | | | | |
| 7% sugar | | | | |
| 7% sugar + 2 ppm | 61.44 | 37.76% to | No | Iso sweet |
| (R)-N-cyclooctyl-2- | | 62.24% | | |
| methylindoline-1- | | | | |
| carboxamide vs. | | | | |
| 8% sugar | | | | |
| 7% sugar + 2 ppm | 77.09 | 37.76% to | Yes | Less sweet |
| (R)-N-cyclooctyl-2- | | 62.24% | | |
| methylindoline-1- | | | | |
| carboxamide vs. | | | | |
| 9% sugar | | | | |
| 7% sugar + 2 ppm | 88.44 | 37.76% to | Yes | Less sweet |
| (R)-N-cyclooctyl-2- | | 62.24% | | |
| methylindoline-1- | | | | |
| carboxamide vs. | | | | |
| 10% sugar | | | | |

From the results it was determined that the 7% sucrose sample cola solution containing 2 ppm of (R)-N-cyclooctyl-2-methylindoline-1-carboxamide, was perceived as significantly more sweet than the 7% sugar cola solution, significantly less sweet than the 9% and 10% sugar cola solutions (the calculated R-index values exceeding the critical value range and p<0.05) and iso sweet to the 8% sugar cola solution.

Because in example 5 a 2 ppm (R)-N-cyclooctyl-2-methylindoline-1-carboxamide solution was perceived as less sweet than 1% sucrose solution, it would be expected that a 7% sucrose sample cola solution containing 2 ppm of (R)-N-cyclooctyl-2-methylindoline-1-carboxamide would be less sweet than an 8% sucrose solution.

However, the results detailed in table II indicate that a 7% sucrose sample cola solution containing 2 ppm of (R)-N-cyclooctyl-2-methylindoline-1-carboxamide is iso sweet with an 8% sucrose solution. Consequently it may be concluded that (R)-N-cyclooctyl-2-methylindoline-1-carboxamide is behaving as a sweetness enhancer.

1. A compound of formula (I) 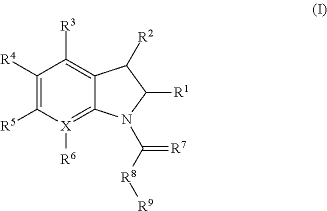
wherein:
X is C,
R¹ to R⁶ may be independently selected from the group consisting of H, C₁-C₃ alkyl, Br, F, and Cl,
R⁷ is O,
R⁸ is NH, and
R⁹ is a group having 5 to 12 carbons selected from the group consisting of cycloalkyl and substituted alkyl, wherein the substituent is selected from the group consisting of aryl and cycloalkyl.

2. A compound according to claim 1 selected from the group consisting of: N-cycloheptylindoline-1-carboxamide; N-cycloheptyl-2-methylindoline-1-carboxamide; N-cyclooctylindoline-1-carboxamide; N-cyclooctyl-2-methyl indoline-1-carboxamide; N-(cyclohexylmethyl)indoline-1-carboxamide; N-(cyclohexylmethyl)-2-methylindoline-1-carboxamide; N-cyclohexyl-3-methylindoline-1-carboxamide; N-cyclohexyl-6-fluoroindoline-1-carboxamide; N-cyclohexyl-5-fluoroindoline-1-carboxamide; N-cyclohexyl-4-methylindoline-1-carboxamide; and N-benzylindoline-1-carboxamide.

3. A composition or consumable product comprising a compound as defined in claim 1.

4. A composition or consumable product comprising a compound as defined in claim 2.

5. A method for creating enhancing or modifying flavour, optionally sweet flavour, of a composition or consumable product comprising adding to said composition at least one compound as defined in claim 1.

6. A method for creating enhancing or modifying flavour, optionally sweet flavour, of a composition or consumable product comprising adding to said composition at least one compound as defined in claim 2.

7. A consumable product as defined in claim 3 comprising the compound at a concentration of 0.01 ppm-500 ppm based on the weight of the consumable product.

8. A consumable product as defined in claim 4 comprising the compound at a concentration of 0.01 ppm-500 ppm based on the weight of the consumable product.

9. A consumable product as defined in claim 3 wherein the consumable product is selected from the group consisting of foodstuffs, confectionary products, baked products, sweet products, savoury products, dairy products, beverages and oral care products.

10. A consumable product as defined in claim 4 wherein the consumable product is selected from the group consisting of foodstuffs, confectionary products, baked products, sweet products, savoury products, dairy products, beverages and oral care products.

11. A method for creating enhancing or modifying flavour, optionally sweet flavour, of a composition or consumable product comprising adding to said composition or consumable product at least one compound as defined in claim 1 selected from the group consisting of (R)-N-cyclooctyl-2-methylindoline-1-carboxamide and (R)-N-cycloheptyl-2-methylindoline-1-carboxamide.

12. A composition or consumable product comprising at least one compound as defined in claim 1 selected from the group consisting of (R)-N-cyclooctyl-2-methylindoline-1-carboxamide and (R)-N-cycloheptyl-2-methylindoline-1-carboxamide.

13. A consumable product comprising the compound as defined in claim 12 at a concentration of 0.01 ppm-500 ppm based on the weight of the consumable product.

14. A consumable product as defined in claim 12 wherein the consumable product is selected from the group consisting of foodstuffs, confectionary products, baked products, sweet products, savoury products, dairy products, beverages and oral care products.

15. A consumable product as defined in claim 13 wherein the consumable product is selected from the group consisting of foodstuffs, confectionary products, baked products, sweet products, savoury products, dairy products, beverages and oral care products.
